(19) United States
(12) Patent Application Publication
(10) Pub. No.: US 2025/0339373 A1
(43) Pub. Date: Nov. 6, 2025

(54) PRODUCTION METHOD OF A COMBINATORIAL DELIVERY SYSTEM OF VINE LEAVES AND PROPOLIS POLYPHENOLS FOR VARIOUS USES

(71) Applicant: APIVITA S.A., Athens (GR)

(72) Inventor: Konstantinos GARDIKIS, ATHENS (GR)

(21) Appl. No.: 18/866,328

(22) PCT Filed: Feb. 16, 2023
(86) PCT No.: PCT/EP2023/053963

§ 371 Date: Nov. 15, 2024

(30) Foreign Application Priority Data
May 16, 2022 (GR) 20220100396

(51) Int. Cl. A61K 9/127; A61K 8/9789; A61K 36/87; A61Q 17/04; A61Q 19/00

(52) U.S. Cl. CPC A61K 9/127; A61K 8/9789; A61K 36/87; A61Q 17/04; A61Q 19/00; A61K 2236/15; A61K 2236/17

(57) ABSTRACT

A production method of a combinatorial delivery system of vine leaves and propolis polyphenols comprising vine leaves in a ratio of 25-50%/25-50%/25-50% asyrtiko/athiri/aidani are used which are chopped into parts <0.8 mm, mixed until completely homogenized dispersed at a rate of 1 kg/min in a container that contains an extraction solvent system, in agitation at 500-3000 rpm, and propolis with a ratio of 75-25% to 50-50%, without prior treatment as long as it contains total polyphenols >1800 mg/l gallic acid equivalent after dissolving 10% per weight of propolis in ethanol, which is crushed into particles <1 mm, after cooling for 24 hours at a temperature of −20° C. and it is dispersed at a rate of 1 kg/min in another container that contains an extraction solvent system, in agitation at 500-3000 rpm they are placed together in a container that has already produced a liposomal system.

### TECHNICAL FIELD

The present invention relates to a production method of a stable system for encapsulating and delivering polyphenols derived from specific varieties of vine leaves and propolis of a specific composition, which offers protection and controlled release of the polyphenols and to its cosmetological use as well as other uses either in its original form or after integration into cosmetic or pharmaceutical formulations, characterized by the fact that the components of the system are extracted and simultaneously encapsulated in liposomes, cyclodextrins as well as combinatorial liposome-cyclodextrin carriers, showing a controlled release of the components.

### TECHNICAL BACKGROUND

Until today, no similar product or similar production method has occurred. In particular, vine leaves consist a byproduct of the vinification procedure. Agricultural byproducts are usually sources of valuable bioactive components. The investigation of the biological value of byproducts is essential for sustainable agriculture but also for the biological properties they can impart to cosmetics, pharmaceuticals and food. In viticulture, vine leaves are a bulky byproduct that is removed and treated as waste in the vinification procedure, with applications limited at best to limited edible products and at worst to the production of a soil conditioner.

Considering its importance in vinification, vine (Vitis vinifera L.) is considered one of the most important fruit cultures in the world, occupying more than 7.5 Mha of the global area (FAO 2016) and producing 80 million tons in 2018 (FAOSTAT 2020). Currently, there are approximately 2000 different varieties of V. vinifera used for grape production worldwide. About 50% of the world's grape production is intended for wine production, an industry of extremely strategic importance for the economy of many countries, with 30% aiming at the commercialization of fresh grapes and the rest being dried as raisin or consumed as grape juice or must.

Due to its wide use, the fruit is the most studied vine product. Vine leaves, although abundant, have been significantly less studied, while it has been shown to contain a wide range of phenolic compounds and antioxidants, which are already used in traditional medicine to treat bleeding, inflammation, diarrhea and liver complications caused by diabetes. Due to their enormous abundance and their non-use—or their use in low-value products—vine leaves consist a byproduct with enormous utilization potential.

Depending on the vine variety, phenolic compounds may vary, while the main bioactive phenolic compounds of the leaves are the flavonoids (+)-catechin, (−)-epicatechin, apigenin, kaempherol, quercetin, myricetin, quercetin-4′-glucoside, and rutin, and the stilbenes cis- and trans-resveratrol monomers, piceid and astringin (Katalinic et al., 2013).

Propolis is a resinous substance produced by bees and which exhibits antimicrobial and antioxidant activity that finds application in the preparation of functional foods and cosmetics as well as in traditional medicine. Depending on the flora of the geographic area of beehives and its collection period, its composition varies. It contains an average of 50% resins, 30% waxes, 10% aromatics, 5% pollen and 5% various other ingredients. The bioactive components of propolis are polyphenols, terpenes, steroids, as well as sugars and amino acids. Its main polyphenols are flavonoids, mainly quercetin, galangin, luteolin, apigenin and phenolic acids, mainly caffeic acid, caffeic acid phenethylester, ferulic acid, p-coumaric acid. The complexity of the propolis structure combinatorial with the different composition depending on the area and season of collection makes both propolis and its extracts extremely difficult to standardize (Stavropoulou et al., 2021).

Polyphenols of both vine and propolis constitute important bioactive molecules with mainly antioxidant, anti-inflammatory and photoprotective effects on the skin (Sangiovanni et al., 2019, Karapetsas et al., 2019, Letsiou et al., 2020). Despite their beneficial properties, both propolis and vine polyphenols present specific challenges that limit their use and effectiveness.

In principle, polyphenol stability against non-enzymatic degradation, during processing and their shelf life are affected by several parameters, such as molecular structure, pH, temperature, oxygen, light, processing conditions and interactions and/or the presence of other compounds and components. In general, polyphenols are unstable molecules and are affected by the extraction conditions where usually either wetting with the extraction solvent for long time intervals is performed, or higher extraction temperatures are used. High temperatures, prolonged times as well as the use of high energy techniques, such as microwave or ultrasound assisted extraction, can lead to degradation of susceptible polyphenols, and degradation of final extract quality. Polyphenols are easily oxidized and can leave black dots when deposited on skin, acquire an unpleasant odor—which limits their topical use—as well as lose their activity due to degradation. (Munin and Edwards-Lévy).

Additionally, propolis and vine leaves polyphenols, being relatively lipophilic, require solvents that exhibit toxicity both per os and topically. The most common solvents used for extraction are various alcohols such as methanol and ethanol. Of these, methanol exhibits the disadvantage that while being an effective solvent for polyphenols, it is also highly toxic. Ethanol is also an effective solvent for polyphenols, but exhibits the disadvantage that it is associated with skin breakdown and local irritations, thus, it is not suitable for local administration—while per os administration shows known toxicity problems.

Apart from extraction and stability related issues, propolis and vine leaves polyphenols present further challenges that limit their use.

A limiting factor for the topical use of polyphenols is the bioavailability of its components, the depth and penetration rate into the epidermis. It is generally accepted that polyphenols from plant material, such as vine leaves, show limited penetration into human skin. (Nichols, J. A., & Katiyar, S. K. 2010). Also, propolis polyphenols, depending on their physicochemical properties and especially the lipophilicity they exhibit, upon local administration are mostly concentrated in the stratum corneum. (Alonso et al. 2014, Abla et al. 2013) while a much smaller percentage penetrates deeper skin layers.

Finally, an additional problem is the duration of contact of polyphenols with skin cells or mucous membranes, especially in cases where prolonged antioxidant protection and/or photoprotective action and/or angiogenic action is required.

To address the above problems, polyphenol protection, bioavailability increase and release rate modification systems have been developed. Techniques such as spray-drying, freeze-drying, extrusion, emulsification, coacervation, molecular encapsulation in cyclodextrins (Grgić et al. 2020), encapsulation in liposomes (Fang and Bhandari, 2010, Figueroa-Robles, et al., 2021) and others.

It is known from the literature that the formation of encapsulation complexes of bioactive substances in cyclodextrins offers advantages such as increased solubility in water, protection from oxidation and protection from degradation of the molecules, and for those reasons complexes of propolis polyphenols with cyclodextrins have been created in the past. Cyclodextrins have been used to encapsulate vine-derived polyphenols, mainly resveratrol, but not vine leaf-derived polyphenols.

Regarding duration of action and bioavailability, controlled release systems involving propolis have been reported (De Luca et al., Franca et al., Balata et al.) which either use ethanol as solvent—with the aforementioned toxicity problems—or synthetic polymers. A controlled release system of propolis polyphenols with natural and non-toxic solvents has been created by our laboratory (WO 2017/089842 A1). Regarding Vitis vinifera, systems that cause control of the release rate of polyphenols have been reported and involve grape seed oil emulsions (Guel et al. 2019) and resveratrol release systems (Song et al., 2021, Paczkowska-Walendowska M, et al., 2021). However, no controlled release systems that encapsulate polyphenols from vine leaves have been reported.

The combination of liposomes and cyclodextrins in a unique system can offer the combinatorial advantages of the two carriers, as described in our laboratory's patent for the creation of a method for the production and stabilization of a controlled release propolis colloidal system (WO2017089842A1 patent).

### SUMMARY OF THE INVENTION

The present invention aims at the beneficial properties of combining vine leaves and propolis polyphenols in a delivery system.

In particular, the invention aims to overcome the disadvantages of the prior art and proposes a production method of a combinatorial delivery system of vine leaves and propolis polyphenols for various uses characterized in that vine leaves are used in a proportion of asyrtiko/athiri/aidani 25-50%/25-50%/25-50% which are chopped into small pieces (<0.8 mm), mixed until completely homogenized and then dispersed at a rate of 1 kg/min in a container that contains an extraction solvent system, which is in agitation at 500-3000 rpm, and propolis with a ratio of 75-25% to 50-50%, without prior treatment as long as it contains total polyphenols >1800 mg/l gallic acid after dissolving 10% per weight of propolis in ethanol, which is crushed into small particles (<1 mm), after cooling for 24 hours at a temperature of −20° C. and then it is dispersed at a rate of 1 kg/min in another container that contains an extraction solvent system, which is in agitation at 500-3000 rpm and then they are placed together in a container that has already produced a liposomal system. The system eliminates the disadvantages related to solubility, stability, penetration depth and duration of action of polyphenols. In particular, in the present invention the simultaneous extraction and encapsulation of polyphenols of the vine leaves of three specific varieties (Assyrtiko, Athiri, Aidani) and propolis with a high content of polyphenols is carried out for the first time, in a combinatorial liposome-cyclodextrin system consisting exclusively of natural and safe components and which provides a controlled release of the encapsulated polyphenols.

More specifically, the present invention utilizes the special characteristics of both carriers: the ability of cyclodextrins to encapsulate polyphenols and the enhancement of their permeability through the skin as well as the ability of liposomes to encapsulate large amounts of components of various degrees of polarity, for local delivery of components and control of their release rate. Encapsulation technology allows component release in 2 phases: a rapid phase where the propolis polyphenols are mainly released and a prolonged phase where the vine leaves polyphenols are mainly released. The procedure of extraction and encapsulation of polyphenols from the two natural products in the combinatorial system takes place in a single container.

The aim of the present invention is the preparation of a system for vine leaves and propolis polyphenols encapsulation and delivery, where the vine leaves and propolis polyphenols are extracted and simultaneously encapsulated in liposome-cyclodextrin combinatorial carriers. The system, when found under suitable conditions, exhibits a controlled release of the components, while under suitable storage conditions, it keeps the components encapsulated and protected in the combinatorial system. The final preparation is suitable for dermatological and other uses, exploiting the bioactive properties of polyphenols, either as such or after integration into cosmetic or pharmaceutical formulations.

According to other optional features of the production method, it can optionally include one or more of the following characteristics alone or in combination:
the vine leaves are collected before spraying and dried in the absence of light to have moisture values <8% and total ash <10%, while each variety should contain total polyphenols >2000 mg/l gallic acid after dissolving 10% per weight in ethanol,
the extraction solvent system consists of deionized water and either vegetable 1,3-propanediol or glycerol in a ratio of 1,3 propanediol (or glycerol)/water: 15/85 to 80/20, while hydroxypropyl-β-cyclodextrin or β-cyclodextrin has been predissolved in the deionized water at a concentration of 2-10% w/w,
the liposomal system consists of 30%-95% Phosphatidylcholine, 4%-10% Phosphatidylserine, 0%-3% Lysophosphatidylcholine, 0%-3% Phosphatidylinositol, 0%-22% cholesterol, 0-10% cholate salts, and their solvent is 1,3 propanediol or glycerol, with a ratio ranging from 20/80 to 80/20% w/w,
the deionized water quality requirement is: <=1 μS/cm at 25° C.,
the vine leaves/cyclodextrin—solvent system is added to the liposomal system at a rate of 10 ml/sec so that the liposomal system obtains a concentration of 1.0-7.5% w/w, and system pH is adjusted in the range of 5.0-8.0 and stirred at 3000 rpm for 2 hours,
the propolis/cyclodextrin system is then added to the stirred container at a rate of 10 ml/sec in a ratio of 10-17%, and after complete addition pH is readjusted in the range of 5.0-8.0 and stirring follows at 4000 rpm for 3 hours,
   at a rate of 100 ml/sec the propolis/cyclodextrin system in a ratio of 5-20% is then added to the container which is now stirred—and for another 10 minutes at 1000 rpm, and after complete addition system pH is finally adjusted to the range of 3.5-6.2, a procedure which results in a part of the propolis-cyclodextrin complex being encapsulated inside the liposomes and a part being free in the suspension, exhibiting a different release rate,
   the mixture is then left in a hermetically sealed container to rest at 5-7° C. for 24 hours and then the system is cold filtered through an array of cartridge filters with pore size of 10 μm-5 μm-1 μm-0.45 μm and the pH is retested which, if necessary, is readjusted in the range of 3.5-6.2
   the system is then allowed to reach room temperature and the mean hydrodynamic diameter of the particles is measured, which should range from 200 nm to 600 nm with a polydispersity index <0.7, while in case it is outside these limits, a second filtration at room temperature through an array of cartridge filters with a pore size of 0.45 μm-0.2 μm follows and then the total polyphenol content is measured which must be >1600 mg/L GAE.
   the release rate of the encapsulated polyphenols in a buffer solution at pH 7.2 at 37° C. is then determined, which is 25-50% in 30 minutes, 50-75% in 8 hours while complete release occurs in 24 hours, and the system is stored in a dark container at a temperature of 5-7° C., where it remains stable for 1 year.
in order to perform an in vitro study of the propolis polyphenol release, a specified amount of the colloid is placed in dialysis bags of MW=1000 molecular exclusion placed in distilled water with pH=7.2 and temperature 37° C. under gentle agitation, where at specific time points samples are taken and their concentration in polyphenols is measured while the amount of water removed is replaced with distilled water with pH=7.2 and temperature 37° C. to maintain tank conditions.

The advantages and innovative elements of the invented method are as follows:
Production of a complex combinatorial system for the administration of polyphenols derived from vine leaves and propolis with a particularly simple preparation method. The procedure of forming encapsulation complexes, between cyclodextrin and polyphenols, as well as the encapsulation of polyphenols in liposomes, either in free form or in complex form with cyclodextrin, is performed during extraction in a single container.
Due to the physicochemical properties of the selected cyclodextrins and phospholipids, almost all (>95%) of the polyphenols, both less and more lipophilic, are encapsulated in a water-soluble system.
The time required for the creation of the colloid is short and the energy supply is minimal, so the final production cost is proportionally kept at very low levels.
The interaction of the two carriers with the polyphenols offers controlled release in suitable conditions—rapid for propolis polyphenols and prolonged for vine leaves polyphenols. In storage conditions the polyphenols are kept protected inside the carriers.
By changing the ratio between phospholipids and cyclodextrins, the polyphenols release rate can be modified, which is very useful depending on the application of the invention.
The combination of the components and the lipid composition of the liposomes are such so as to avoid fusion and liposomes aggregation issues resulting in excellent stability of the system both from a physicochemical point of view (mean hydrodynamic diameter, z-potential, polydispersity index, pH) and from the side of the retention of polyphenols inside the liposomes during storage.
Only safe and skin and mucous membrane friendly components are used.
The final extract is titrated to total polyphenols.
The system is self-preserving for a period of 1 year at a temperature of 5-7° C. and does not require the addition of preservatives, despite the large percentage of water it contains. This is due to the high content of polyphenols that exhibit, among others, antimicrobial properties.
The addition of a chelating agent is not required, due to the quality of the water used.
The sensitive polyphenols of the system are protected from oxidation or degradation due to light and temperature increase because of the double encapsulation in liposomes and cyclodextrins. They are also protected for the same reason from interaction with other components in case the system is used as a component of cosmetic or pharmaceutical formulations.
The system can be used directly on human skin and/or mucous membrane or integrated into cosmetic or pharmaceutical formulations for topical or systemic administration. In cosmetic or pharmaceutical formulations, it even provides transparent liquids in case it is required, e.g. in jellies preparation.
The system can be used with appropriate formulation as a food supplement, pharmaceutical formulation, nutraceutical and as a functional food ingredient.

The foregoing and other objects, features and advantages of the present invention will become more apparent from the following detailed description when taken in conjunction with the accompanying drawings in which:

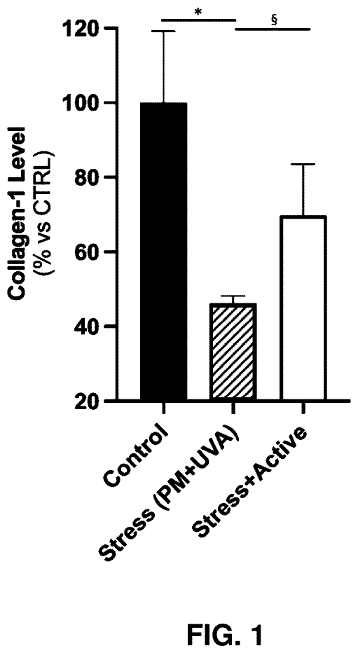

FIG. 1: is a Collagen-I quantification bar graph by specific signal detection from cell count and in the control group (*p<0.05 § p<0.1—t-test for binary comparisons against the stress group; “control” or “CTRL”: cell not exposed to UVA and not treated with the propolis and vine leaves polyphenols combinatorial delivery system, “Stress”: Cells exposed to stresses but not treated with polyphenols combinatorial delivery system “Stress+Active”: Cells exposed to stresses and treated with polyphenols combinatorial delivery system).

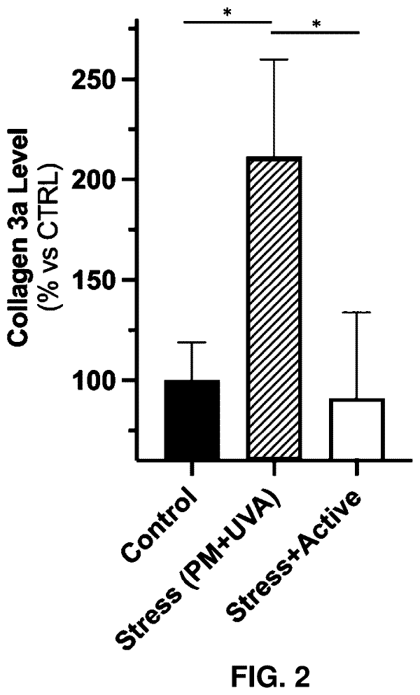

FIG. 2: is a Collagen-III quantification bar graph by specific signal detection from cell count and in the control group (*p<0.05, t-test for binary comparisons against the stress group; “control” or “CTRL”: cell not exposed to UVA and not treated with the propolis and vine leaves polyphenols combinatorial delivery system, “Stress”: Cells exposed to stresses but not treated with polyphenols combinatorial delivery system “Stress+Active”: Cells exposed to stresses and treated with polyphenols combinatorial delivery system).

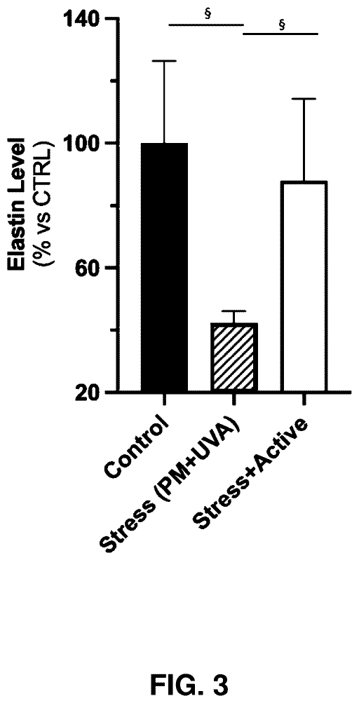

FIG. 3: is a Elastin quantification bar graph by specific signal detection from cell count and in the control group (§ p<0.1, t-test for binary comparisons against the stress group; “control” or CTRL: cell not exposed to UVA and not treated with the propolis and vine leaves polyphenols combinatorial delivery system, “Stress”: Cells exposed to stresses but not treated with polyphenols combinatorial delivery system “Stress+Active”: Cells exposed to stresses and treated with polyphenols combinatorial delivery system).

### DISCLOSURE OF THE INVENTION

In order for our invention to be understood by those skilled in the art, we proceed then to the detailed description of the preparation method of the vine leaves and propolis polyphenol delivery system in liposome/cyclodextrin combinatorial carriers.

Vine leaves are harvested before spraying and dried in the absence of light to show moisture values <8% and total ash <10%. Each variety preferably contain total polyphenols >2000 mg/l gallic acid equivalents after dissolving 10% per weight in ethanol and subsequent measurement in a spectrophotometer using the Folin-Ciocalteu method.

Propolis can be used without prior treatment, as long as it contains, preferably, total polyphenols >1800 mg/l gallic acid equivalents after dissolving 10% per weight of propolis in ethanol and following measurement in a spectrophotometer by the Folin-Ciocalteu method. The proportion of vine leaves and propolis ranges from 75%/25% to 50%/50%.

Deionized water quality requirement is: <=1 μS/cm at 25° C., which exceeds the specification of the European Pharmacopoeia for the preparation of parenteral medicinal products. This quality is necessary for the complete absence of charges in the final formulation that could lead the lipid membranes of the liposomes to aggregation and ultimately to a decrease in the product stability.

The production of deionized water used in our invention is performed as follows:

The water from the water supply network enters the raw water tank (volume 2 m³), with a suitable pumping assembly, passes through an automatic turbidity filter to remove turbidity and solid particles and activated carbon to remove chlorine and organic load and then an antiscalant is dosed to bind its hardness. Before entering the central assembly of the reverse osmosis unit, it passes through a 1 micron cartridge filter.

The finely processed water for use in reverse osmosis enters the reverse osmosis unit of 350 It/h capacity with 70% recovery. The produced water of the unit is tanked in a stainless steel tank with a volume of 5 m³. From this tank, the water with a suitable pumping assembly feeds the deionizer and is driven on-line to the extraction tank via U.V. To avoid stagnant water in the network, there is a continuous water recirculation with return to the tank.

In order to prepare the system, at first, the vine leaves in a proportion of asyrtiko/athiri/aidani 25-50%/25-50%/25-50% are chopped into small parts (<0.8 mm) and mixed until completely homogenized. They are then dispersed at a rate of 1 kg/min into the solvent system, which is in agitation at 500-3000 rpm. The concentration of vine leaves can be from 1.8 to 8% per weight depending on their content in polyphenols. The extraction solvent system consists of deionized water and either vegetable 1,3 propanediol or glycerol in a ratio of 1,3 propanediol (or glycerol)/water: to 80/20. Hydroxypropyl-β-cyclodextrin or β-cyclodextrin is predissolved in the deionized water at a concentration of 2-10% w/w.

The propolis is crushed into small particles (<1 mm) after cooling it for 24 hours at −20° C. In a second container, the crushed propolis is dispersed at a rate of 1 kg/min into the solvent system, which is in agitation at 500-3000 rpm. Propolis concentration can be from 1 to 12% per weight depending on its content in polyphenols and depending on the release rate desired by the system. The extraction solvent system consists of deionized water and either vegetable 1,3-propanediol or glycerol at a ratio of 1,3-propanediol (or glycerol)/water: 15/85 to 80/20. Hydroxypropyl-β-cyclodextrin or β-cyclodextrin is predissolved in the deionized water at a concentration of 2-10% w/w.

The production of the liposomal system takes place in a third container. The liposomal system consists of:
30%-95% Phosphatidylcholine
4%-10% Phosphatidylserine
0%-3% Lysophosphatidylcholine
0%-3% Phosphatidylinositol
0%-22% cholesterol
0-10% cholate salts

Their solvent is 1,3 propanediol or glycerol. The ratio of the above components to 1,3 propanediol or glycerol ranges from 20/80 to 80/20% w/w.

The vine leaves/cyclodextrin-solvent system is then added to the liposomal system at a rate of 10 ml/sec so that the liposomal system obtains a concentration of 1.0-7.5% w/w. After complete addition, the system pH is adjusted to the range of 5.0-8.0 and stirred at 3000 rpm for 2 hours.

The propolis/cyclodextrin system is then added to the stirred container at a rate of 10 ml/sec in a ratio of 10-17%. After complete addition the system pH is readjusted to the range of 5.0-8.0 followed by stirring at 4000 rpm for 3 hours.

Finally, at a rate of 100 ml/sec the propolis/cyclodextrin system in a ratio of 5-20% is added to the container which is now stirred—and for another 10 minutes at 1000 rpm. After complete addition the system pH is finally adjusted to the range of 3.5-6.2. With the procedure of these steps, it is ensured that a part of the propolis-cyclodextrin complex will be encapsulated inside the liposomes and a part will be free in the suspension. These parts exhibit the characteristic of different release rate.

The mixture is then left in a hermetically sealed container to rest at 5-7° C. for 24 hours. Then, the system is cold filtered through an array of cartridge filters with pore size of 10 μm-5 μm-1 μm-0.45 μm and the pH is retested which, if necessary, is readjusted in the range of 3.5-6.2.

The system is allowed to reach room temperature and the mean hydrodynamic diameter of the particles is measured which should range from 200 nm to 600 nm with a polydispersity index <0.7. In case the values are outside the above limits, a second filtration at room temperature through an array of cartridge filters with a pore size of 0.45 μm-0.2 μm follows.

If the mean hydrodynamic diameter and polydispersity index values are within specifications, the total polyphenol content is measured. Though the method according to the invention allows to obtain a combination of liposomes and cyclodextrins which is particularly effective in e.g. protecting, increasing bioavailability and modulating release rate of phenolic compounds, it should be considered that the highest the concentration in phenolic compounds is, the more effective the combinatorial delivery system will be in providing the biological effects as reported in the example section. In a particular embodiment the total polyphenol of the combinatorial delivery system of the invention content is superior to 1600 mg/L gallic acid equivalents (GAE).

Determining gallic acid equivalents (GAE) content is a well-known method of estimating the content in total phenolic compounds of a composition. Gallic acid equivalents can be determined, for example, using the method of Folin-Ciocalteu method as described by Chun and Kim (2004).

The release rate of polyphenols is then determined in a buffer solution at pH 7.2 at 37° C. and the system is stored in a dark container at a temperature of 5-7° C., where it remains stable for 1 year.

The release of polyphenols (cumulative release) at pH 7.2 and at a temperature of 37° C. is 25-50% in 30 minutes and 50-75% in 8 hours, while the system releases practically all the encapsulated polyphenols in 24 hours.

The determination of the mean hydrodynamic diameter and the Polydispersity index is performed via Dynamic Light Scattering.

The in vitro study of propolis polyphenol release is performed with the help of dialysis bags:

Specified amount of the colloid is placed in dialysis bags of MW=1000 molecular exclusion. The bag is placed in distilled water with pH=7.2 and temperature 37° C. under gentle agitation. At specific time points, samples are taken and their concentration in polyphenols is measured while the amount of water removed is replaced with distilled water with pH=7.2 and temperature 37° C. to maintain the tank conditions.

In order to make the present invention fully understood, we proceed to the following examples:

### Example 1

Vine leaves in a ratio of assyrtiko/athiri/aidani varieties 34/33/33% w/w are used. The chopped leaves are dispersed in a ratio of 8% at a rate of 1 kg/min in a solvent system consisting of deionized water vegetable 1,3 propanediol 75/25. As the system is under intense agitation (3000 rpm) and at a temperature of 20° C., it is added to a liposomal suspension so that the latter obtains a per weight proportion of 6.0%. Hydroxypropyl-β-cyclodextrin has been predissolved in deionized water at a concentration of 4.5% w/w.

Propolis in a separate container is dispersed in a solvent system with the same ratio as above up to a concentration of 10% w/w. The propolis/cyclodextrin-solvent system is added at a rate of 10 ml/sec the propolis/cyclodextrin system at a ratio of 6% to the first container. After complete addition, system pH is adjusted to 5.0, followed by stirring at 4000 rpm for 3 hours.

Then, at a rate of 100 ml/sec, the propolis/cyclodextrin system at a ratio of 15% is added to the container which is now stirred—and for another 10 minutes at 1000 rpm. After complete addition system pH is finally adjusted to 4.0.

The procedure as stated in the disclosure of the invention follows.

The final suspension after filtration exhibits the values shown in table 1.

TABLE 1

| Parameter | Value | |
|---|---|---|
| Mean hydrodynamic particle diameter | 250-320 | nm |
| Polydispersity index | <0.60 | |
| Total polyphenols (gallic acid equivalents) | 2150 | mg/L |

| | |
|---|---|
| Cumulative release of polyphenols in 30 min % | 30-35 |
| Cumulative release of polyphenols in 8 hours % | 70-80 |

### Example 2

Vine leaves in a ratio of assyrtiko/athiri/aidani varieties 50/35/15% w/w are used. The chopped leaves are dispersed in a ratio of 8% at a rate of 1 kg/min in a solvent system consisting of deionized water vegetable 1,3 propanediol 75/25. As the system is under intense agitation (3000 rpm) and at a temperature of 20° C., it is added to a liposomal suspension so that the latter obtains a per weight proportion of 6.0%. Hydroxypropyl-β-cyclodextrin has been predissolved in deionized water at a concentration of 4.5% w/w.

Propolis in a separate container is dispersed in a solvent system with the same ratio as above up to a concentration of 10% w/w. The propolis/cyclodextrin-solvent system is added at a rate of 10 ml/sec the propolis/cyclodextrin system at a ratio of 10% to the first container. After complete addition, system pH is adjusted to 5.0, followed by stirring at 4000 rpm for 3 hours.

Then, at a rate of 100 ml/sec, the propolis/cyclodextrin system at a ratio of 20% is added to the container which is now stirred—and for another 10 minutes at 1000 rpm. After complete addition system pH is finally adjusted to 4.0.

The procedure as stated in the disclosure of the invention follows.

The final suspension after filtration exhibits the values shown in table 2.

The determination of the mean hydrodynamic diameter and the Polydispersity index is performed via Dynamic Light Scattering.

TABLE 2

| Parameter | Value | |
|---|---|---|
| Mean hydrodynamic particle diameter | 20-300 | nm |
| Polydispersity index | <0.70 | |
| Total polyphenols (gallic acid equivalents) | 1600 | mg/L |

| | |
|---|---|
| Cumulative release of polyphenols in 30 min % | 45-50 |
| Cumulative release of polyphenols in 8 hours % | 40/45 |

### Experimental Evidence of the Propolis and Vine Leaves Polyphenols Combinatorial Delivery System Use

In order to document the use of the propolis and vine leaves polyphenols combinatorial delivery system (hereinafter referred to as Active) in cosmetic forms we performed an in vitro study to test the effectiveness against pollution (environmental pollution and UVA).

The induced regulation of collagen-I, collagen-IIIa and elastin was assessed in human dermal fibroblasts via in situ immunofluorescence microscopy analysis.

The system was diluted in the culture medium just before treatment with fibroblasts taking a freshly prepared solution to the desired final concentration (0.1%).

Human dermal fibroblasts were plated in a 96-well plate at an initial density of 10,000 cells/well and maintained under optimal growth conditions for 2 days. On day 1, particles (ERMCZ100, similar to PM10—PAHs; 1 μg/cm2) were added to the cells belonging to “Stress” groups and “Stress+Active” groups. After 1 hour of contact the cells were irradiated with UVA (LED source, 365 nm, 3 J/cm2). Immediately after irradiation, the culture medium was renewed, Active was added to cells (0.1%) and left in contact for 24 h. The control group received culture medium renewal only. After 24 hours of incubation, cells were fixed on the plate and analyzed by in situ immunofluorescence detection of Collagen-I, Collagen-IIIa and elastin.

Cells were incubated with primary antibodies specific for the biomarker of interest (Table 3) in a BSA solution in PBS. Excess primary antibodies were removed by a series of washing steps, cells were then incubated with the secondary antibody conjugated to a fluorophore (Table 3), and nuclei were labeled using DAPI (4′,6-diamidine-2-phenylindole). Finally, antibodies and excess DAPI were removed by a series of washing steps with PBS.

TABLE 3

| Primary antibody | Secondary antibody |
|---|---|
| Rabbit polyclonal to Collagen I | Goat anti-Rabbit AlexaFluor 647 |
| Abcam (ab 34710) | Invitrogen (A21244) |
| Rabbit polyclonal to Collagen III | Goat anti-Rabbit AlexaFluor 647 |
| Abcam (ab7778) | Invitrogen (A21244) |
| Mouse monoclonal to Elastin | Goat anti-mouse AlexaFluor 647 |
| Abcam (ab9519) | Invitrogen (A21235) |

### Biomarkers and Antibodies

A series of images per biomarker was acquired with an epifluorescence microscope (ThermoFisher, Evos M5000) using strictly the same acquisition time and resolution (40× objective) per biomarker. Images were collected and recorded at all levels of fluorescence signal intensity (0 to 65535; 16-bit .TIFF format), then analyzed using ImageJ software (Rasband, NIH).

For each biomarker, quantification was performed on each image by integrating the specific fluorescence signal above the intensity threshold and then normalized to the evaluation surface and the number of cells (calculated from the DAPI cell nuclei detection images).

An effectiveness value (%) was obtained for the experimental groups considering the “stress” group at minimum yield (0%) and the untreated group (control) at maximum yield (at 100%).

As illustrated in FIGS. 1-3, the presence of active components:
causes increased levels of collagen-I compared to the stress condition (44% effectiveness), (§: p<0.1; *: p<0.05),
counteracts stress-induced changes in collagen-IIIa levels (100% effectiveness) (*: p<0.05),
causes increased levels of elastin compared to the stress condition (79% effectiveness) (§ p<0.1).

The obtained results suggest that the tested active presents beneficial effects on collagen-I, collagen-IIIa and elastin in contrast to the changes caused by exposure to stress (pollution) in skin cells.

### REFERENCES

FAO and OIV, FAO-OIV FOCUS 2016 Table and dried grapes, Paris, 2016, ISBN 978-92-5-109708-3 (FAO), ISBN 979-10-91799-74-4 (OIV), 62 pp
FAOSTAT. 2020. Available online: http://www.fao.org/faostat/en/#data/QC.
Katalinic V, Smole Mozina S, Generalic I, Skroza D, Ljubenkov I, Klancnik A. Phenolic Profile, Antioxidant Capacity, and Antimicrobial Activity of Leaf Extracts from Six Vitis vinifera L. Varieties, International Journal of Food Properties, 2013
Sangiovanni E, Di Lorenzo C, Piazza S, Manzoni Y, Brunelli C, Fumagalli M, Magnavacca A, Martinelli G, Colombo F, Casiraghi A, Melzi G, Marabini L, Restani P, Dell'Agli M. Vitis vinifera L. Leaf Extract Inhibits In Vitro Mediators of Inflammation and Oxidative Stress Involved in Inflammatory-Based Skin Diseases. Antioxidants (Basel). 2019
Letsiou S, Kapazoglou A, Tsaftaris A. Transcriptional and epigenetic effects of Vitis vinifera L. leaf extract on UV-stressed human dermal fibroblasts. Mol Biol Rep. 2020 August; 47(8):5763-5772.
Munin A, Edwards-Lévy F: Encapsulation of Natural Polyphenolic Compounds; a Review. Pharmaceutics 2011
Alonso C, Rubio L, Touriño S, Martí M, Barba C, Fernández-Campos F, Coderch L, Parra J L: Antioxidative effects and percutaneous absorption of five polyphenols. Free Radic Biol Med. 2014
Abla M J, Banga A K: Quantification of skin penetration of antioxidants of varying lipophilicity. Int J Cosmet Sci. 2013
Karapetsas, A., Voulgaridou, G. P., Konialis, M., Tsochantaridis, I., Kynigopoulos, S., Lambropoulou, M., Stavropoulou, M. I., Stathopoulou, K., Aligiannis, N., Bozidis, P., Goussia, A., Gardikis, K., Panayiotidis, M. I., & Pappa, A. Propolis Extracts Inhibit UV-Induced Photodamage in Human Experimental In Vitro Skin Models. Antioxidants (Basel, Switzerland), 8(5), 125. 2019.
De Luca M P, Franca J R, Augusto F, Macedo F, Grenho L, Cortes M E, Faraco G A, Moreira A N, Vagner R. Santos V R: Propolis Varnish: Antimicrobial Properties against Cariogenic Bacteria, Cytotoxicity and Sustained-Release Profile. BioMed Research International. 2014
Balata G, El Nahas H M, Radwan S: Propolis organogel as a novel topical delivery system for treating wounds: Drug Delivery. 21(1):55-61. 2014
Franca J R, De Luca M P, Ribeiro T G, Castilho R O, Moreira A N, Santos V R, Faraco A A: Propolis-based chitosan varnish: drug delivery, controlled release and antimicrobial activity against oral pathogen bacteria. MC Complement Altern Med. 12(14):478. 2014
Nichols, J A, & Katiyar, S K. Skin photoprotection by natural polyphenols: anti-inflammatory, antioxidant and DNA repair mechanisms. Archives of dermatological research, 302(2), 71-83. 2010.
Grgić, J, Šelo, G, Planinić, M, Tišma, M, & Bucić-Kojić, A Role of the Encapsulation in Bioavailability of Phenolic Compounds. Antioxidants (Basel, Switzerland), 9(10), 923. 2020
Fang Z, Bhandari B, Encapsulation of polyphenols—a review, Trends in Food Science & Technology, Volume 21, Issue 10, 2010
Figueroa-Robles A, Antunes-Ricardo M, Guajardo-Flores D, Encapsulation of phenolic compounds with liposomal improvement in the cosmetic industry, International Journal of Pharmaceutics, Volume 593, 2021
Song J, Zong J, Ma C, Chen S, Li H, Zhang D. Microparticle prepared by chitosan coating on the extruded mixture of corn starch, resveratrol, and α-amylase controlled the resveratrol release. Int J Biol Macromol. 31; 185:773-781. 2021
Estévez M, Güell C, De Lamo-Castellví S, Ferrando M. Encapsulation of grape seed phenolic-rich extract within W/O/W emulsions stabilized with complexed biopolymers: Evaluation of their stability and release. Food Chem. 2019 Jan. 30; 272:478-487. doi: 10.1016/j.foodchem.2018.07.217. Epub 2018 Aug. 1. PMID: 30309571.
Paczkowska-Walendowska M, Dvořák J, Rosiak N, Tykarska E, Szymańska E, Winnicka K, Ruchała M A, Cielecka-Piontek J. Buccal Resveratrol Delivery System as a Potential New Concept for the Periodontitis Treatment. Pharmaceutics. 2021 Mar. 20; 13(3):417. doi: 10.3390/pharmaceutics13030417. PMID: 33804630; PMCID: PMC8003728.
WO 2017/089842 A1 Inventors: Gardikis Konstantinos, Koutsianas Nikolaos, Patera Anna, Dragani Panagiota, Tsoukalas Anagnostis-Ioannis, Letsiou Sofia, Method for Preparing a Stable Controlled-Release Propolis Colloidal Dispersion System for Various Uses
Tao Y, Wang D, Hu Y, Huang Y, Yu Y, Wang D: The immunological enhancement activity of propolis flavonoids liposome in vitro and in vivo. Evid Based Complement Alternat Med. 2014; 2014:483513
Yuan J, Lu Y, Abula S, Hu Y, Liu J, Fan Y, Zhao X, Wang D, Liu X, Liu C: Optimization on preparation condition of propolis flavonoids liposome by response surface methodology and research of its immunoenhancement activity. Biomacromolecules. 11; 14(3):854-61. 2013
Stavropoulou M I, Stathopoulou K, Cheilari A, Benaki D, Gardikis K, Chinou I, Aligiannis N. NMR metabolic profiling of Greek propolis samples: Comparative evaluation of their phytochemical compositions and investigation of their anti-ageing and antioxidant properties. J Pharm Biomed Anal. 5; 194:113814. 2021
Ock Kyoung Chun, Dae-Ok Kim, Consideration on equivalent chemicals in total phenolic assay of chlorogenic acid-rich plums, Food Research International, Volume 37, Issue 4, 2004, Pages 337-342.

1. A production method of a combinatorial delivery system of vine leaves and propolis polyphenols, comprising:
a step of chopping vine leaves in a proportion of asyrtiko/athiri/aidani varieties 25-50%/25-50%/25-50% w/w, wherein the vine leaves are chopped into pieces less than 0.8 mm,
a step of mixing the chopped vine leaves until completely homogenized, and then
a step of dispersion at a rate of 1 kg/min in a first container that contains an extraction solvent system comprising deionizing water, with an agitation at 500-3000 rpm,
a step of cooling propolis for 24 hours at a temperature of −20° C.,
a step of crushing propolis into particles less than 1 mm,
a step of dissolving 10% per weight of propolis in ethanol without prior treatment as long as it contains total polyphenols more than 1800 mg/l gallic acid equivalents after dissolving, and then it is dispersed at a rate of 1 kg/min in a second container that contains an extraction solvent system comprising deionizing water, which is in agitation at 500-3000 rpm, and then
a step of placement carried out in a third container that has already produced a liposomal system, the placement step comprising placement together of vine leaves system and propolis system with a ratio of 75-25% to 50-50%.

2. A production method of a combinatorial delivery system of vine leaves and propolis polyphenols according to claim 1, further comprising a step of collecting the vine leaves before a step of spraying and drying in the absence of light to have moisture values less than 8% and total ash less than 10%, while each variety contains total polyphenols more than 2000 mg/l gallic acid equivalents after a step of dissolving 10% per weight in ethanol.

3. A production method of a combinatorial delivery system of vine leaves and propolis polyphenols according to claim 1, wherein the extraction solvent system comprises deionized water and either vegetable 1,3-propanediol or glycerol in a ratio of 1,3 propanediol (or glycerol)/water: 15/85 to 80/20, while hydroxypropyl-β-cyclodextrin or β-cyclodextrin has been predissolved in deionized water of the extraction solvent at a concentration of 2-10% w/w.

4. A production method of a combinatorial delivery system of vine leaves and propolis polyphenols, according to claim 1, wherein the liposomal system comprises 30%-95% Phosphatidylcholine, 4%-10% Phosphatidylserine, 0%-3% Lysophosphatidylcholine, 0%-3% Phosphatidylinositol, 0%-22% cholesterol, 0-10% cholate salts, and their solvent is 1,3 propanediol or glycerol, with a ratio ranging from 20/80 to 80/20% w/w.

5. A production method of a combinatorial delivery system of vine leaves and propolis polyphenols, according to claim 3, wherein the deionized water quality is: less than or equal to 1 μS/cm at 25° C.

6. A production method of a combinatorial delivery system of vine leaves and propolis polyphenols, according to claim 1, characterized in that said further comprising at least one of:
a step of adding the vine leaves in the solvent system with cyclodextrin to the liposomal system at a rate of 10 mL/sec so that the liposomal system obtains a concentration of 1.0-7.5% w/w,
a step of adjusting the system pH in the range of 5.0-8.0 and
a step of stirring at 3000 rpm for 2 hours; and
a step of adding the propolis in the system solvent with cyclodextrin to the stirred second container at a rate of 10 ml/sec in a ratio of 10-17%, and after complete addition,
a step of adjusting pH in the range of 5.0-8.0, and
a step of stirring at 4000 rpm for 3 hours; and
a step of adding at a rate of 100 ml/sec the propolis/cyclodextrin solvent system in a ratio of 5-20% to the third container which is stirred,
a step of stirring for another 10 minutes at 1000 rpm, and after complete addition,
a step of adjusting pH of the system to the range of 3.5-6.2, which results in a part of the propolis-cyclodextrin complex being encapsulated inside the liposomes and a part being free in the suspension, exhibiting a different release rate,
a step of leaving the mixture in a hermetically sealed container at 5-7° C. for 24 hours, and then
a step of cold filtering the system through an array of cartridge filters with pore size of 10 μm, then 5 μm, then 1 μm and then 0.45 μm, and
a step of retesting the pH, if necessary, the pH is readjusted in the range of 3.5-6.2, the system is then allowed to reach room temperature; and
a step of measuring a mean hydrodynamic diameter of the particles, wherein if said mean hydrodynamic diameter is outside a range from 200 nm to 600 nm with a polydispersity index less than 0.7,
a step of a second filtering at room temperature through an array of cartridge filters with a pore size of 0.45 μm and then 0.2 μm follows, and then
a step of measuring total polyphenol content which must be more than 1600 mg/L gallic acid equivalents,
a step of determining a release rate of encapsulated polyphenols in a buffer solution at pH 7.2 at 37° C., which is 25-50% in 30 minutes, 50-75% in 8 hours while complete release occurs in 24 hours, and
a step of storage the system in a dark container at a temperature of 5-7° C.

7. A production method of a combinatorial delivery system of vine leaves and propolis polyphenols, according to claim 1, further comprising a step of in vitro study of the propolis polyphenol release, comprising:
the placement of a specified amount of a colloid in dialysis bags of MW=1000 molecular exclusion placed in distilled water with pH=7.2 and temperature 37° C. under gentle agitation,
taking of samples at specific time points, and
measurement of their concentration in polyphenols while the water removed is replaced with distilled water with pH=7.2 and temperature 37° C. to maintain container conditions.

8. A cosmetic, a nutraceutical or a pharmaceutical composition comprising the combinatorial delivery system of vine leaves and propolis polyphenols obtained from the method of claim 1.

9. A method of using the cosmetic, nutraceutical or pharmaceutical composition according to claim 8, comprising applying said composition to counteract stress-induced changes in skin cells.

10. A method of using the a cosmetic, neutraceutical or pharmaceutical composition according to claim 8, comprising applying said composition to protect skin cells against pollution.

11. A method of using a combinatorial delivery system of vine leaves and propolis polyphenols obtained from the method of claim 1, comprising applying said combinatorial delivery system on human skin and/or mucous membrane.

12. A method of using a combinatorial delivery system of vine leaves and propolis polyphenols obtained from the method of claim 1, comprising applying said combinatorial delivery system to protect skin cells against UVA and/or against microparticles.

13. A method of using a combinatorial delivery system of vine leaves and propolis polyphenols obtained from the method of claim 1, comprising applying said combinatorial delivery system to protect skin cells against pollution.
